(19) United States
(12) Patent Application Publication
(10) Pub. No.: US 2010/0233219 A1
(43) Pub. Date: Sep. 16, 2010

(54) INORGANIC NANOPARTICLE COMPROMISING AN ACTIVE SUBSTANCE IMMOBILIZED ON THE SURFACE AND A POLYMER

(75) Inventors: Makiko Aimi, Kanagawa (JP); Ryoichi Nemori, Kanagawa (JP); Masayoshi Kojima, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/293,392

(22) PCT Filed: Mar. 30, 2007
(86) PCT No.: PCT/JP2007/057719

§ 371 Date: Sep. 17, 2008

(30) Foreign Application Priority Data
Mar. 30, 2006 (JP) 2006-093480

(51) Int. Cl. A61K 8/02; B32B 5/16; A61K 9/32; A61Q 19/02; A61K 49/08; C12P 21/02; A23L 1/30

(52) U.S. Cl. 424/401; 428/407; 424/501; 426/531; 424/62; 424/9.3; 435/68.1; 426/72

(57) ABSTRACT

An object of the present invention is to provide highly safe nanoparticles which can be used simultaneously for imaging, hyperthermia and DDS and have high drug incorporation ratio. The present invention provides a nanoparticle which comprises an inorganic nanoparticle of 1 to 500 nm in average particle size having an active substance immobilized on the surface and a polymer.

### TECHNICAL FIELD

The present invention relates to a nanoparticle for use in the fields of life science and medical diagnosis. More particularly, the present invention relates to a nanoparticle comprising an inorganic nanoparticle having an active substance immobilized on the surface thereof and a polymer.

### BACKGROUND ART

Fine particle materials have been expected to be widely used in biotechnology. Recently, in particular, fine nanoparticle materials developed through advancement of nanotechnology have been actively studied to be applied in biotechnology and medical treatment and a lot of research reports have been made. Of such fine particle materials, magnetic fine particle materials have been widely used in the field of biotechnology. For example, magnetic fine particles on which an antibody or the like is immobilized are used for immunodiagnosis. Also, magnetic fine particles having DNA immobilized on the surface are used in gene engineering in a broad range including separation of mRNA or single strand DNA and separation of DNA binding protein. Moreover, magnetically responsive particles are very effective for analyzing protein interaction which is one of the important subjects in proteome analysis.

Such particles are also useful in the field of medical diagnosis, for example, as a contrast medium in MRI diagnosis and in hyperthermia of cancer. Cancer cells are killed when heated to 42.5° C. or higher (e.g., Non-Patent Document 1). In present hyperthermia, since normal tissues and tumor tissues are heated without distinction, tissues are heated only to about 42.5° C. at which normal tissues are not so affected in consideration of the burden on patients. However, obviously, the higher the heating temperature, the more cancer cells are killed. Therefore, theoretically, any type of cancer cells would be killed if tumor tissues alone could be specifically heated without heating normal tissues. Hyperthermia with respect to inductive heating using magnetite (Fe₃O₄) which is a magnetically responsive particle as a heating element has been developed and so far produced successful results in regression of tumors in various animals (mice, rats, hamsters, rabbits) and cancers (brain tumor, skin cancer, tongue cancer, breast cancer, liver cell cancer, osteosarcoma) (e.g., Non-Patent Document 2 and Non-Patent Document 3).

Since magnetically responsive particles have a small particle size of nanosize, they have significantly improved dispersibility and molecular recognition in an aqueous solution compared to micron-size magnetic particles and latex beads conventionally used. Accordingly, simple replacement of magnetic fine particles or latex carriers used in conventional methods leads to significant increase in sensitivity and shortening of measurement time.

On the other hand, in the field of drug delivery systems (DDS) where great expectations have risen for nanoparticles long before, nanoparticles are quite promising as carriers for drugs and genes. While targeting, which is to make drugs act only on cancer cells or cancer lesions, is required in order to improve therapeutic efficiency of anticancer agents, such nanoparticles can noninvasively lead a certain substance to a site in vivo or make the substance stay there topically utilizing their magnetic characteristics.

Kato et al. have developed ethyl cellulose microcapsules having a diameter of about 250 μm in which mitomycin C and ferrite magnetic powder are encapsulated (hereinafter FM-MMC-mc). In an experiment of treating a VX tumor transplanted in the lower leg of a rabbit, a remarkable antitumor effect has been found in the FM-MMC-mc magnetic leading group in contrast to the MMC normal dosage-form administration group. This is because MMC has been released to neighboring tumor tissues from capsules magnetically accumulated in small arteries in tumors over a long period. The result suggests a strong possibility of intensive targeting therapy which has not been available in conventional methods (e.g., Non-Patent Document 4). However, having a size as large as 250 μm, FM-MMC-mc cannot reach minute portions such as blood capillaries.

Patent Document 1 discloses a metal oxide complex comprising metal oxide particles having a particle size of 5 to 200 nm dispersed at least on the surface of a gel. Patent Document 2 discloses a natural polymer powder containing noble metal nanoparticles. Patent Document 3 discloses water dispersible nanoparticles containing a semiconductive material or a metallic material. However, since these particles do not contain an active substance (drug), they have no DDS function.

Patent Document 4 discloses a drug targeting system using nanoparticles prepared from a polymer material. Patent Document 5 discloses a nanoparticle formulation of a medicinal or cosmetic active substance having a core/shell structure. Patent Document 6 discloses a spherical protein particle having a particle size of 1 μm or more in the form of a composition containing a drug. Since these system, formulation and particle do not contain magnetically responsive particles, nanoparticles cannot be magnetically led to diseased sites.

Also, Patent Document 7 discloses a process for preparing a nanoparticle coated with magnetic metal oxide. Patent Document 8 discloses a metal or a semiconductor atom bonded to a plurality of sugar nanoparticle ligands.

### [Non-Patent Document 1] Dewey, W. C., Radiology, 123, 463-474 (1977)

[Non-Patent Document 2] Kobayashi, T., Jpn. J. Cancer Res., 89, 463-469 (1998)

### [Non-Patent Document 3] Kobayashi, T., Melanoma Res., 13, 129-135 (2003)

### [Non-Patent Document 4] Tetsuro Kato, Enhanced Effect of Antitumor Agent by Magnetic Leading of Microcapsules, Japanese Journal of Cancer and Chemotherapy, 8(5), 698-706, 1981

### [Patent Document 1] Japanese Patent Laid-Open No. 2000-256015

### [Patent Document 2] Japanese Patent Laid-Open No. 2004-244433

### [Patent Document 3] Japanese Publication of International Application No. 2004-517712

### [Patent Document 4] Japanese Publication of International Application No. 2001-502721

### [Patent Document 5] Japanese Publication of International Application No. 2002-531492

### [Patent Document 6] Japanese Publication of International Application No. 2005-500304

### [Patent Document 7] Japanese Publication of International Application No. 2002-517085

### [Patent Document 8] Japanese Publication of International Application No. 2004-511511

### DISCLOSURE OF THE INVENTION

An object of the present invention is to provide highly safe nanoparticles which can be used simultaneously for imaging, hyperthermia and DDS and have high drug incorporation ratio.

The present inventors have conducted intensive studies to solve the above problems and as a result, have found that nanoparticles which can solve the above problem can be produced by mixing inorganic nanoparticles having an active substance immobilized on the surface and a polymer such as protein, and the present invention has been completed.

Accordingly, the present invention provides a nanoparticle which comprises an inorganic nanoparticle of 1 to 500 nm in average particle size having an active substance immobilized on the surface and a polymer.

Preferably, the inorganic nanoparticle is a magnetic nanoparticle.

Preferably, the inorganic nanoparticle is iron oxide, ferrite, zinc oxide, titanium oxide, silica or alumina.

Preferably, an active substance is immobilized through physical adsorption on the surface of the inorganic nanoparticle having amino acid immobilized on the surface.

Preferably, amino acid is immobilized on the surface of the inorganic nanoparticle surface-modified with a compound represented by the formula:

R¹—(OCH(R²)CH₂)ₙ—O-L-X

wherein R¹ represents an alkyl or alkenyl group having a carbon chain length between of 1 and 20 inclusive or an unsubstituted phenyl group or phenyl group substituted with an alkyl or alkoxyl group having a carbon chain length of 10 or less; R² represents a hydrogen atom or methyl group; n represents an integer of 1 to 20; L represents a single bond or an alkylene group having 1 to 10 carbon atoms; and X represents a carboxylic acid group, a phosphoric acid group, a sulfonic acid group or a boric acid group, and further, an active substance is immobilized through physical adsorption on the surface.

Preferably, the nanoparticle of the present invention has an average particle size of 10 to 1000 nm.

Preferably, the inorganic nanoparticle has an average particle size of 1 to 50 nm.

Preferably, 0.1 to 100% by weight of the inorganic nanoparticle is contained with respect to the polymer.

Preferably, 0.1 to 100% by weight of the active substance is contained with respect to the polymer.

Preferably, the active substance is a cosmetic ingredient, a functional food ingredient or a pharmaceutical ingredient.

Preferably, the cosmetic ingredient is a moisturizer, a skin-whitening agent or an anti-aging agent, the functional food ingredient is vitamin or an antioxidant, and the pharmaceutical ingredient is an anticancer agent, an antiallergic agent, an antithrombotic agent or an antiinflammatory agent.

Preferably, the polymer is a synthetic polymer, a biodegradable polymer or a natural polymer.

Preferably, the polymer is protein.

Preferably, the protein is crosslinked during or after preparing the nanoparticle.

Preferably, the protein is crosslinked by adding 0.1 to 100% by weight of a crosslinking agent with respect to the weight of the protein.

Preferably, an inorganic or organic crosslinking agent or enzyme may be used as a crosslinking agent. Specific examples of inorganic or organic crosslinking agents include, but not limited to, chromium salts (chrome alum, chromium acetate, etc.); calcium salts (calcium chloride, calcium hydroxide, etc.); aluminum salts (aluminum chloride, aluminum hydroxide, etc.); dialdehydes (glutaraldehyde, etc.); carbodiimides (EDC, WSC, N-hydroxy-5-norbornene-2,3-dicarboxylmide (HONB), N-hydroxysuccinic acid imide (HOSu), dicyclohexylcarbodiimide (DCC), etc.); N-hydroxysuccinimide; and phosphorus oxychloride.

While the enzyme is not particularly limited as long as it has action for crosslinking protein, preferably transglutaminase is used.

Preferably, the protein is crosslinked in an organic solvent.

Preferably, the protein has a lysine residue and a glutamine residue.

Preferably, the protein is collagen, gelatin, albumin, ovalbumin, casein, transferrin, fibrin, fibrinogen or a mixture thereof.

Preferably, the protein is acid-treated gelatin or albumin.

Preferably, the protein is acid-treated gelatin, and the nanoparticle is prepared by crosslinking the acid-treated gelatin with an enzyme during or after preparing the nanoparticle comprising the inorganic nanoparticle and the acid-treated gelatin.

Preferably, the nanoparticle of the present invention is produced through the following steps:

(a) mixing a solution of at least one active substance and inorganic nanoparticles, thereby adsorbing the active substance to the surface of the inorganic nanoparticles;
(b) dissolving protein in an aqueous medium;
(c) mixing the inorganic nanoparticles to which at least one active substance is adsorbed and the protein solution;
(d) pouring the solution prepared in step (c) into an organic solvent; and
(e) crosslinking the protein by adding a crosslinking agent.

Preferably, the nanoparticle of the present invention is produced through the following steps:

(a) mixing a solution of at least one active substance and inorganic nanoparticles, thereby adsorbing the active substance to the surface of the inorganic nanoparticles;
(b) dissolving protein in an aqueous medium;
(c) mixing the inorganic nanoparticles to which at least one active substance is adsorbed and the protein solution;
(d) adding an enzyme; and
(e) pouring the solution prepared in step (d) into an organic solvent to crosslink the protein with the enzyme.

Preferably, the nanoparticle of the present invention is obtained by treating protein with a reducing agent to break a disulfide bond in protein molecules, then forming nanoparticles of the protein, and further treating the protein with an oxidant.

Preferably, in the step of treating protein with an oxidant, protein nanoparticles dispersed in an organic solvent are treated with the oxidant.

Preferably, the protein is albumin, ovalbumin, transferrin or globulin.

Preferably, the nanoparticle of the present invention is produced through the following steps:

(a) mixing a solution of at least one active substance and inorganic nanoparticles, thereby adsorbing the active substance to the surface of the inorganic nanoparticles;
(b) dissolving protein whose disulfide bond is reduced in water;
(c) mixing the inorganic nanoparticles to which at least one active substance is adsorbed and the protein solution;
(d) pouring the solution prepared in step (c) into an organic solvent; and
(e) treating the resultant with an oxidant.

Preferably, the protein is casein.

Preferably, the nanoparticle of the present invention is produced through the following steps:

(a) mixing a solution of at least one active substance and inorganic nanoparticles, thereby adsorbing the active substance to the surface of the inorganic nanoparticles;
(b) dissolving casein in a basic aqueous medium at pH 8 or more;
(c) mixing the inorganic nanoparticles to which at least one active substance is adsorbed and the casein solution; and
(d) pouring the solution prepared in step (c) into an aqueous medium at pH 3.5 to 7.5.

Preferably, the nanoparticle of the present invention is produced through the following steps:

(a) mixing a solution of at least one active substance and inorganic nanoparticles, thereby adsorbing the active substance to the surface of the inorganic nanoparticles;
(b) dissolving casein in a basic aqueous medium at pH 8 or more;
(c) mixing the nanoparticles to which at least one active substance is adsorbed and the casein solution; and
(d) lowering the pH of the solution prepared in step (c) to pH 3.5 to 7.5 while stirring.

Preferably, 0.1 to 100% by weight of lipid is added with respect to the weight of the polymer.

Preferably, 0.1 to 100% by weight of a cationic or anionic polysaccharide is added with respect to the weight of the polymer.

Preferably, 0.1 to 100% by weight of a cationic or anionic protein is added with respect to the weight of the polymer.

Preferably, 0.1 to 100% by weight of cyclodextrin is added with respect to the weight of the polymer.

An another aspect of the present invention provides a hyperthermia agent comprising the nanoparticle of the present invention.

A still another aspect of the present invention provides an MRI contrast medium comprising the nanoparticle of the present invention.

A still another aspect of the present invention provides a drug delivery agent comprising the nanoparticle of the present invention.

A still another aspect of the present invention provides a method for producing a nanoparticle comprising an inorganic nanoparticle of 1 to 500 nm in average particle size having an active substance immobilized on the surface, and a protein, the method comprising crosslinking the protein during and/or after preparing the nanoparticle.

Preferably, the protein is crosslinked by an enzyme.

Preferably, the protein is crosslinked by the enzyme in an organic solvent.

A still another aspect of the present invention provides a method for producing a nanoparticle comprising an inorganic nanoparticle of 1 to 500 nm in average particle size having an active substance immobilized on the surface, and a protein, the method comprising treating the protein with a reducing agent to break a disulfide bond in protein molecules, then forming nanoparticles of the protein, and further treating the protein with an oxidant.

A still another aspect of the present invention provides a method for producing a nanoparticle comprising an inorganic nanoparticle of 1 to 500 nm in average particle size having an active substance immobilized on the surface, and a protein, the method comprising treating the protein with a reducing agent to break a disulfide bond in protein molecules, then forming nanoparticles of the protein, and further treating the protein dispersed in an organic solvent with an oxidant.

A still another aspect of the present invention provides a method for producing a nanoparticle of 10 to 1000 nm in average particle size, comprising an inorganic nanoparticle of 1 to 500 nm in average particle size having an active substance immobilized on the surface and casein, the method comprising the following steps:

(a) mixing a solution of at least one active substance and inorganic nanoparticles, thereby adsorbing the active substance to the surface of the inorganic nanoparticles;
(b) dissolving casein in a basic aqueous medium at pH 8 or more;
(c) mixing the inorganic nanoparticles to which at least one active substance is adsorbed and the casein solution; and
(d) pouring the solution prepared in step (c) into an aqueous medium at pH 3.5 to 7.5.

A still another aspect of the present invention provides a method for producing a nanoparticle of 10 to 1000 nm in average particle size, comprising an inorganic nanoparticle of 1 to 500 nm in average particle size having an active substance immobilized on the surface and casein, the method comprising the following steps:

(a) mixing a solution of at least one active substance and inorganic nanoparticles, thereby adsorbing the active substance to the surface of the inorganic nanoparticles;
(b) dissolving casein in a basic aqueous medium at pH 8 or more;
(c) mixing the inorganic nanoparticles to which at least one active substance is adsorbed and the casein solution; and
(d) lowering the pH of the solution prepared in step (c) to pH 3.5 to 7.5 while stirring.

### BEST MODE FOR CARRYING OUT THE INVENTION

Embodiments of the present invention are described in detail below.

The nanoparticle of the present invention comprises an inorganic nanoparticle of 1 to 500 nm in average particle size having an active substance immobilized on the surface, and a polymer.

The nanoparticle of the present invention has an average particle size of generally 1 to 1000 nm, preferably 10 to 1000 nm, more preferably 30 to 500 nm, particularly preferably 50 to 200 nm. Having a particle size of nano order as described above, the nanoparticle of the present invention can reach minute portions such as blood capillaries.

The inorganic nanoparticle used in the present invention has an average particle size of 1 to 500 nm, preferably 1 nm to 50 nm, more preferably 1 nm to 30 nm.

In the present invention, while the active substance is immobilized on the surface of the inorganic nanoparticle, immobilization herein described may be performed by physical adsorption or chemical adsorption. Types of adsorption include, but not limited to, adsorption by ion interaction, adsorption by hydrophobic interaction, and adsorption by coordinate bonds.

The dispersion of inorganic nanoparticles used in the present invention can be prepared, for example, by adding an aqueous solution of a surfactant (e.g., polyoxyethylene(4,5)lauryl ether acetate) to agglomerates of inorganic nanoparticles and dispersing the mixture. However, the method of preparing the dispersion of inorganic nanoparticles is not limited thereto. For example, a hydrophilic polymer [polyethylene glycol, sodium polyphosphate, etc.] or phospholipid (phosphatidylcholine, etc.) may coexist when or after synthesizing inorganic nanoparticles.

The nanoparticle of the present invention contains preferably 0.1 to 100% by weight of inorganic nanoparticles with respect to the weight of a polymer such as protein.

Examples of inorganic nanoparticles used in the present invention include, but not limited to, iron oxide nanoparticles, zinc oxide nanoparticles, titanium oxide nanoparticles, silica nanoparticles and alumina nanoparticles. Preferred examples thereof include magnetically responsive particles.

Any magnetically responsive particle may be used as the magnetically responsive particle used in the present invention as long as the particle absorbs electromagnetic waves and generates heat, and is harmless to humans. In particular, particles which absorb electromagnetic waves with a frequency hardly absorbed to humans and generate heat are preferably used. Preferred examples of magnetically responsive particles include iron, platinum, iron oxide and ferrite (Fe,M)₃O₄, and iron oxide particles are particularly preferred. Herein, iron oxides include, in particular, Fe₃O₄ (magnetite), γ-Fe₂O₃ (maghemite) and intermediates and mixtures thereof. Also, such magnetically responsive particles may have a core-shell structure in which compositions on the surface and in the inside are different. In the formula, M represents a metal ion capable of forming magnetic metal oxide when used together with the iron ion. Such a metal ion is typically selected from transition metals, most preferably Zn²⁺, Co²⁺, Mn²⁺, Cu²⁺, Ni²⁺ and Mg²⁺. The molar ratio M/Fe is determined with respect to the stoichiometric composition of ferrite to be selected.

In the present invention, inorganic nanoparticles which were surface-modified with a compound represented by the following formula are preferably used.

R¹—(OCH(R²)CH₂)ₙ—O-L-X Formula

wherein R¹ represents an alkyl or alkenyl group having a carbon chain length between of 1 and 20 inclusive or an unsubstituted phenyl group or phenyl group substituted with an alkyl or alkoxyl group having a carbon chain length of 10 or less; R² represents a hydrogen atom or methyl group; n represents an integer of 1 to 20; L represents a single bond or an alkylene group having 1 to 10 carbon atoms; and X represents a carboxylic acid group, a phosphoric acid group, a sulfonic acid group or a boric acid group.

Examples of alkyl groups having a carbon chain length between of 1 and 20 inclusive include a methyl group, an ethyl group, a n-propyl group, an isopropyl group, a t-butyl group, an octyl group and a cetyl group. Examples of alkenyl groups having a carbon chain length between of 1 and 20 inclusive include the above alkyl groups having at least one double bond.

Specific examples of compounds represented by the above formula include the followings, but the compound in the present invention is not limited thereto.

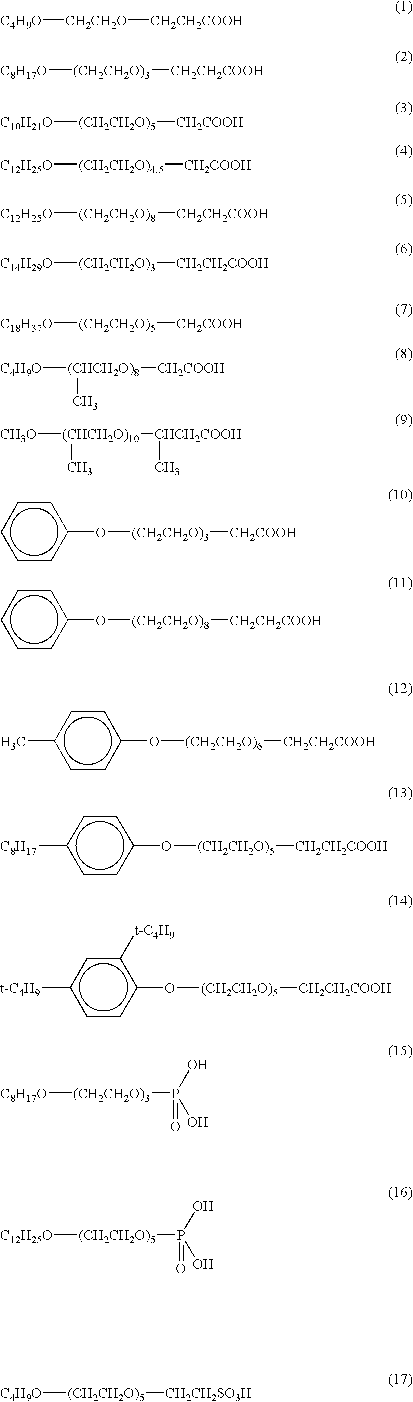

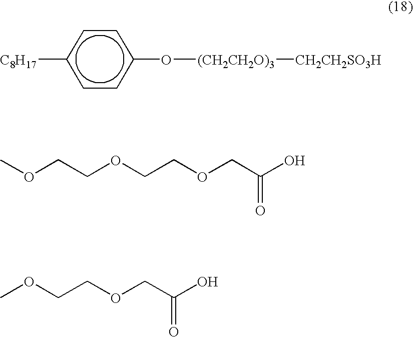

Preferably 1 to 200, more preferably 1 to 100 molecules of amino acid may be immobilized per inorganic nanoparticle used in the present invention. Examples of amino acids to be immobilized include glycine, alanine, valine, leucine, isoleucine, norvaline, norleucine, serine, threonine, aspartic acid, glutamic acid, asparagine, glutamine, lysine, arginine, cysteine, methionine, ornithine, citrulline, phenylalanine, tyrosine, tryptophan, histidine, β-alanine, γ-aminobutyric acid (GABA) and proline. Water-soluble amino acids are preferred as immobilized amino acids, which may be selected from, for example, glycine, alanine, serine, threonine, aspartic acid, glutamic acid, lysine, arginine, cysteine, proline, β-alanine and GABA.

The inorganic nanoparticle having amino acid immobilized on the surface can be prepared, for example, by irradiating inorganic nanoparticles having an average particle size of 1 to 50 nm dispersed in water with ultrasonic wave in the presence of amino acid. Ultrasonic irradiation for immobilizing amino acid on the surface of inorganic nanoparticles may be performed by a method known to those skilled in the art, for example, by using a commercially available ultrasonic bath. Preferably, ultrasonic irradiation may be performed in a buffer, for example, a phosphate buffer, at pH 5.0 or more. The time for ultrasonic irradiation is generally 1 minute to 2 hours, which is not particularly limited and may be accordingly determined as long as amino acid can be immobilized on the surface of magnetic nanoparticles. Also, preferably ultrasonic wave with a high frequency output of 0.1 to 200 W is irradiated.

An active substance is immobilized on the surface of the inorganic nanoparticle used the present invention. The active substances used in the present invention include cosmetic ingredients such as moisturizing agents, skin-whitening agents and anti-aging agents, functional food ingredients such as vitamins and antioxidants and pharmaceutical ingredients such as anticancer agents, antiallergic agents, antithrombotic agents and antiinflammatory agents.

Specific examples of moisturizing agents used in the present invention include, but are not limited to, hyaluronic acid, ceramide, Lipidure, isoflavone, amino acid, and collagen.

Specific examples of skin-whitening agents used in the present invention include, but are not limited to, vitamin C, arbutin, hydroquinone, kojic acid, Lucinol, and ellagic acid.

Specific examples of anti-aging agents used in the present invention include, but are not limited to, retinoic acid, retinol, vitamin C, kinetin, β-carotene, astaxanthin, and tretinoin.

Specific examples of an antioxidant that can be used in the present invention include, but are not limited to, vitamin C derivative, vitamin E, kinetin, α-lipoic acid, and coenzyme Q10.

Specific examples of an anticancer agent that can be used in the present invention include, but are not limited to, pyrimidine fluoride antimetabolites (e.g., 5-fluorouracil (5FU), tegafur, doxifluridine, and capecitabine), antibiotics (e.g., mitomycin (MMC) and Adriacin (DXR)), purine antimetabolites (e.g., folic acid antimetabolites such as methotrexate, and mercaptopurine), vitamin A active metabolites (e.g., antimetabolites such as hydroxy carbamide, tretinoin, and tamibarotene), molecular targeting agents (e.g., Herceptin and imatinib mesylate), platinum drugs (e.g., Briplatin and Randa (CDDP), Paraplatin (CBDC), Elplat (Oxa), and Aqupla), plant alkaloids (e.g., Topotecin, Campto (CPT), Taxol (PTX), Taxotere (DTX), and Etoposide), alkylating agents (e.g., Busulfan, cyclophosphamide, and Ifomide), antiandrogens (e.g., bicalutamide and flutamide), female hormones (e.g., Fosfestrol, chlormadinone acetate, and estramustine phosphate), LH-RH agonists (e.g., Leuplin and Zoladex), antiestrogens (e.g., tamoxifen citrate and toremifene citrate), aromatase inhibitors (e.g., fadrozole hydrochloride, anastrozole, and Exemestane), progestins (e.g., medroxyprogesterone acetate), and BCG.

Specific examples of antiallergic agents used in the present invention include, but are not limited to: mediator antireleasers, such as disodium cromoglycate and tranilast; histamine H1 antagonists, such as ketotifen fumarate and azelastine hydrochloride; thromboxane inhibitors, such as ozagrel hydrochloride; leukotriene antagonists, such as pranlukast; and suplatast tosylate.

The active substance used in the present invention may be used alone or in combination of two or more types.

In the present invention, ultrasonic irradiation for immobilizing an active substance on the surface of inorganic nanoparticles may be performed by a method known to those skilled in the art, for example, by using a commercially available ultrasonic bath. Ultrasonic irradiation is performed, for example, in water. The time for ultrasonic irradiation is generally 1 minute to 2 hours, which is not particularly limited and may be accordingly determined as long as an active substance can be immobilized on the nanoparticle surface. Also, preferably ultrasonic wave with a high frequency output of 0.1 to 200 W is irradiated.

Types of polymers used in the present invention are not particularly limited, and a synthetic polymer or a natural polymer may be used. Although biodegradable polymers are preferred, the polymer is not limited thereto.

Examples of synthetic polymers used in the present invention include, but not limited to, polyether, polyamine, polyacrylate, polymethacrylate, polycyanoacrylate, polyarylamide, polylactate, polyglycolate, polyanhydride, polyorthoester, polystyrene, polyvinyl, polyacrolein, polyglutaraldehyde, and derivatives, copolymers and mixtures thereof. Polyethylene glycol, polyvinyl alcohol, polylactic acid, polyvinyl pyrrolidone and polyalginic acid are preferred.

Examples of biodegradable polymers used in the present invention include, but not limited to, polylactic acid, polyglycolic acid and copolymers thereof.

Examples of natural polymers used in the present invention include protein and polysaccharide.

Among the aforementioned polymers used in the present invention, protein is particularly preferred.

A protein having a molecular weight of approximately 10000 to 1,000,000 is preferably used in the present invention, although types of proteins are not particularly limited. Although the origin of protein is not particularly limited, collagen, gelatin, acid-treated gelatin, albumin, globulin, casein, transferrin, fibrin or fibrinogen can be used. A protein of human origin is particularly preferably used.

In the present invention, gene recombinant gelatin may be used. Since the gene recombinant gelatin is excellent in biocompatibility and non-infectivity and is uniform as compared with natural gelatin and its sequence has been determined, its strength and degradation property can be precisely designed by crosslinking and the like as mentioned below.

As the gene recombinant gelatin, those described in EP 1014176A2 and U.S. Pat. No. 6,992,172 can be used, but the gelatin is not limited thereto.

The biopolymer may be partially hydrolyzed.

The amino acid homology between the gelatin and natural collagen is preferably 40% or more, more preferably 50% or more, more preferably 80% or more, most preferably 90% or more.

The collagen may be any natural collagen, and is preferably type I, type II, type III, type IV or type V collagen. More preferably, the collagen is type I, type II or type III collagen. In another embodiment, the origin of the collagen is preferably human, bovine, swine, mouse, or rat, and is more preferably human.

The isoelectric point of the gene recombinant gelatin is generally 5 to 10, preferably 6 to 10, more preferably 7 to 9.

The gene recombinant gelatin has GXY region which is characteristic of collagen, and its molecular weight is preferably 2 kDa to 100 kDa, more preferably 2.5 kDa to 95 kDa, more preferably 5 kDa to 90 kDa, most preferably 10 kDa to 90 kDa.

Preferably, the gene recombinant gelatin is not deaminated.

Preferably, the gene recombinant gelatin does not contain procollagen and precollagen.

Preferably, the gene recombinant gelatin is a substantially pure collagen material which was prepared by a nucleic acid which encodes a natural collagen.

The protein nanoparticle of the present invention can be prepared according to the methods described in Japanese Patent Laid-Open No. 6-79168 or by C. Coester, Journal of Microencapsulation, 2000, vol. 17, p. 187-193. Preferably, crosslinking agents described in the present specification are used instead of glutaraldehyde.

While the protein in the nanoparticle of the present invention may or may not be crosslinked, preferably the protein is crosslinked. More preferably, the protein is crosslinked during or after preparing the nanoparticle. The protein may be crosslinked by a crosslinking agent or by reducing a disulfide bond in protein molecules and re-bonding after forming particles. In the present invention, protein may be crosslinked by one crosslinking method or in combination of two or more crosslinking methods.

In the present invention, protein is preferably crosslinked in an organic solvent. Water-soluble organic solvents such as ethanol, isopropanol, acetone and THF are preferred as the organic solvent herein used.

When using a crosslinking agent, protein is crosslinked by adding preferably 0.1 to 100% by weight of the crosslinking agent with respect to the weight of the protein.

An inorganic or organic crosslinking agent or enzyme may be used as a crosslinking agent. Specific examples of inorganic or organic crosslinking agents include, but not limited to, chromium salts (chrome alum, chromium acetate, etc.); calcium salts (calcium chloride, calcium hydroxide, etc.); aluminum salt (aluminum chloride, aluminum hydroxide, etc.); carbodiimides (EDC, WSC, N-hydroxy-5-norbornene-2,3-dicarboxylmide (HONB), N-hydroxysuccinic acid imide (HOSu), dicyclohexylcarbodiimide (DCC), etc.); N-hydroxysuccinimide; and phosphorus oxychloride. While the enzyme is not particularly limited as long as it has action for crosslinking protein, preferably transglutaminase is used. Specific examples of protein which is enzymatically crosslinked by transglutaminase are not particularly limited as long as the protein contains a lysine residue or a glutamine residue. Of them, acid-treated gelatin, collagen and albumin are preferred.

Transglutaminase may be originated in mammals or microorganisms. Specific examples thereof include ACTIVA products available from AJINOMONO CO., INC. and transglutaminase originated in mammals sold as a reagent, e.g., transglutaminase originated in guinea pig liver, transglutaminase originated in goat and transglutaminase originated in rabbits available from Oriental Yeast Co., Ltd., Upstate USA Inc. and Biodesign International.

The amount of the crosslinking agent used in the present invention is accordingly determined with respect to types of proteins. Typically, about 0.1 to 100% by weight, preferably about 1 to 50% by weight of the crosslinking agent may be added with respect to the weight of the protein.

Although the time for the crosslinking reaction may be accordingly determined with respect to types of proteins and the size of nanoparticles, the time is usually 1 hour to 72 hours, preferably 2 hours to 24 hours.

Although the temperature in the crosslinking reaction may be accordingly determined with respect to types of proteins and the size of nanoparticles, the temperature is usually 0° C. to 80° C., preferably 25° C. to 60° C.

The crosslinking agent used in the present invention may be used alone or in combination of two or more.

When treating protein with a reducing agent to break a disulfide bond in protein molecules, then forming nanoparticles of the protein and further treating the protein with an oxidant, treating the protein with an oxidant leads to reformation of a disulfide bond between protein molecules and partial reformation of a disulfide bond in the molecules, whereby protein nanoparticles are crosslinked and become insoluble in water. In such cases, although types of proteins used in the present invention are not particularly limited as long as the protein has a disulfide bond, protein having a molecular weight of about 10,000 to 1,000,000 is preferably used. Although the origin of protein is not particularly limited, protein of human origin is preferably used. Of them, albumin, globulin and transferrin are preferred.

Specific examples of reducing agents used in the present invention include dithiothreitol, thioglycolic acid, thioglycolate such as ammonium thioglycolate, cysteine, cysteic acid salt such as cysteine hydrochloride, cysteine derivatives such as N-acetylcysteine and glutathione, thioglycolic acid monoglycerol, cysteamine, thiolactic acid, sulfite, bisulfite and mercaptoethanol. However, the reducing agent in the present invention is not limited to these compounds.

The reducing agent used in the present invention may be used alone or in combination of two or more.

The amount of the reducing agent used in the present invention is accordingly determined with respect to types of proteins. Typically, about 0.1 to 100% by weight, preferably about 1 to 50% by weight of the reducing agent may be added with respect to the weight of the protein.

Although the time for the reduction reaction for treating protein with a reducing agent may be accordingly determined with respect to types of proteins and the size of nanoparticles, the time is usually 5 minutes to 72 hours, preferably 10 minutes to 12 hours.

Although the temperature in the reduction reaction may be accordingly determined with respect to types of proteins and the size of nanoparticles, the temperature is usually 0° C. to 80° C., preferably 25° C. to 40° C.

Specific examples of oxidants used in the present invention include oxygen, hydrogen peroxide, bromate such as sodium bromate and potassium bromate, perborate and sodium percarbonate. However, the oxidant in the present invention is not limited to these compounds. Oxygen in the air may be used as oxygen. More specifically, when using oxygen as the oxidant, protein is treated with oxygen by stirring a dispersion containing nanoparticles in the air. The oxidant used in the present invention may be used alone or in combination of two or more.

The amount of the oxidant used in the present invention is accordingly determined with respect to types of proteins. Typically about 0.1 to 100% by weight, preferably about 1 to 50% by weight of the oxidant may be added with respect to the weight of the protein.

Although the time for the oxidation reaction for treating protein with an oxidant may be accordingly determined with respect to types of proteins and the size of nanoparticles, the time is usually 5 minutes to 72 hours, preferably 10 minutes to 12 hours.

Although the temperature in the oxidation reaction may be accordingly determined with respect to types of proteins and the size of nanoparticles, the temperature is usually 0° C. to 80° C., preferably 25° C. to 60° C.

In a preferred embodiment of the present invention, casein may be used as protein. The origin of casein used in the present invention is not particularly limited. Casein may be originated in milk or beans and α-casein, β-casein, γ-casein, κ-casein or a mixture thereof may be used. The casein may be used alone or in combination of two or more types.

A method for producing the casein nanoparticle of the present invention includes a method comprising dissolving casein in a basic aqueous medium solution of pH 8 or more and injecting the resulting solution into an aqueous medium of pH 3.5 to 7.5, and a method comprising dissolving casein in a basic aqueous medium solution of pH 8 or more and decreasing the pH of the resulting solution to pH 3.5 to 7.5 while stirring the solution.

Preferably, the method comprising dissolving casein in a basic aqueous medium solution of pH 8 or more and injecting the resulting solution into an aqueous medium of pH 3.5 to 7.5 is performed by use of a syringe, because of the simplicity of its operation. However, the method is not particularly limited as long as it satisfies an injection rate, solubility, a temperature, and stirring state. In general, the solution can be injected at an injection rate of 1 mL/min to 100 mL/min. The temperature of the basic aqueous medium can be set appropriately and can be normally 0° C. to 80° C., preferably 25° C. to 70° C. The temperature of the aqueous medium can be set appropriately and can be normally 0° C. to 80° C., preferably 25° C. to 60° C. A stirring speed can be set appropriately and can be normally 100 rpm to 3000 rpm, preferably 200 rpm to 2000 rpm.

Preferably, the method comprising dissolving casein in a basic aqueous medium solution of pH 8 or more and decreasing the pH of the resulting solution to pH 3.5 to 7.5 while stirring the solution is performed by the dropping of an acid, because of the simplicity of its operation. However, the method is not particularly limited as long as it satisfies solubility, a temperature, and stirring state. The temperature of the basis aqueous medium can be set appropriately and can be normally 0° C. to 80° C., preferably 25° C. to 70° C. A stirring speed can be set appropriately and can be normally 100 rpm to 3000 rpm, preferably 200 rpm to 2000 rpm.

Water, a physiological saline, or an aqueous solution or buffer solution of an organic acid or base or an inorganic acid or base can be used as the aqueous medium used in the present invention.

Specific examples thereof include, but not limited to, aqueous solutions using organic acids such as citric acid, ascorbic acid, gluconic acid, carboxylic acid, tartaric acid, succinic acid, acetic acid, phthalic acid, trifluoroacetic acid, morpholinoethanesulfonic acid, and 2-[4-(2-hydroxyethyl)-1-piperazinyl]ethanesulfonic acid; organic bases such as tris(hydroxymethyl), aminomethane, and ammonia; inorganic acids such as hydrochloric acid, perchloric acid, and carbonic acid; and inorganic bases such as sodium phosphate, potassium phosphate, calcium hydroxide, sodium hydroxide, potassium hydroxide, and magnesium hydroxide.

The concentration of the aqueous medium used in the present invention is preferably approximately 10 mM to approximately 1 M, more preferably approximately 20 mM to approximately 200 mM.

The pH of the basic aqueous medium used in the present invention is preferably 8 or higher, more preferably 8 to 11, even more preferably 10 to 11. A pH lower than 8 does not allow the dissolution of casein.

The pH of the acidic aqueous medium used in the present invention is preferably 3.5 to 7.5, more preferably 4 to 6. A pH higher than 7.5 outside the range results in the dissolution of the particle, whereas a pH not higher than 3 tends to increase the particle size.

Specific examples of lipid used in the present invention are listed below. However, in the present invention, the lipid is not limited to these compounds. It includes phosphatidylcholine (lecithin), phosphatidylethanolamine, phosphatidylserine, phosphatidylinositol, phosphatidylglycerol, diphosphatidylglycerol, sphingosines, ceramide, oleic acid, linoleic acid, linolenic acid, palmitic acid, myristic acid, stearic acid, soybean oil, olive oil, and squalane.

The term “anionic polysaccharides” that is used in the present invention refers to polysaccharides having acidic polar groups such as carboxyl, sulfate or phosphate groups. Specific examples thereof include, but are not limited to, chondroitin sulfate, dextran sulfate, carboxymethyl dextran, alginic acid, pectin, carragheenan, fucoidan, agaropectin, porphyran, karaya gum, gellan gum, xanthan gum, and hyaluronic acids.

The term “cationic polysaccharides” used in the present invention refers to polysaccharides having basic polar groups such as amino groups. Specific examples thereof include, but are not limited to, polysaccharides comprising glucosamine or galactosamine as a constitutive monosaccharide such as chitin or chitosan.

The term “anionic proteins” used in the present invention refers to proteins and lipoproteins whose isoelectric points are more basic than the physiological pH. Specific examples thereof include, but are not limited to, polyglutamic acid, polyaspartic acid, lysozyme, cytochrome C, ribonuclease, trypsinogen, chymotrypsinogen, and α-chymotrypsin.

The term “cationic proteins” used in the present invention refers to proteins and lipoproteins whose isoelectric points are more acidic than the physiological pH. Specific examples thereof include, but are not limited to, polylysine, polyarginine, histone, protamine, and ovalbumin.

Specific examples of cyclodextrin used in the present invention are listed below. However, in the present invention, the cyclodextrin is not limited to these compounds. It includes α-cyclodextrin, β-cyclodextrin, γ-cyclodextrin, 2,6-di-O-methyl-α-cyclodextrin, 2,6-di-O-methyl-β-cyclodextrin, glucuronyl glucosyl-β-cyclodextrin, heptakis(2,6-di-O-methyl)-β-cyclodextrin, 2-hydroxyethyl-β-cyclodextrin, hydroxypropyl-β-cyclodextrin, 6-O-α-maltosyl-α-cyclodextrin, methyl-β-cyclodextrin, 2,3,6-tri-O-methyl-β-cyclodextrin, and 6-O-α-D-glucosyl-α-cyclodextrin.

When the inorganic nanoparticle in the present invention is magnetically responsive, the nanoparticle can be magnetically led to a predetermined site. More specifically, the nanoparticle of the present invention can be magnetically led to a diseased site when administered to the body. Also, the nanoparticle led to the diseased site as described above can be observed in MRI imaging. In short, the nanoparticle of the present invention is useful as a contrast medium for MRI.

Further, the nanoparticle of the present invention can release a pharmaceutically active substance encapsulated in the nanoparticle by heating by applying high frequency radiation after the nanoparticle is led to the diseased site according to the above method. In short, the nanoparticle of the present invention is useful as a drug delivery agent.

Further, the nanoparticle of the present invention can also be used as a probe for analytical diagnosis. More specifically, the magnetic nanoparticle can be used for detection, analysis, concentration and purification of amino acid receptors (glutamic acid receptors, aspartic acid receptors and serine receptors).

Although the method for administering the nanoparticle of the present invention is not particularly limited, preferably the nanoparticle is administered via percutaneous absorption or transmucosal absorption, or to the blood vessel, body cavity or lymph by injection. In particular, intravenous injection is preferred.

While the dose of the nanoparticle of the present invention can be accordingly determined based on the body weight of the patient and the condition of the disease, the nanoparticle can be administered in an amount of generally about 10 μg to 100 mg/kg, preferably about 20 μg to 50 mg/kg per dose.

The present invention is described in more detail with reference to Examples below, but the present invention is not limited thereto.

### EXAMPLES

### Preparation Example 1

### Preparation of Magnetically Responsive Particle Dispersion

10.8 g of iron chloride (III) hexahydrate and 6.4 g of iron chloride (II) tetrahydrate were each dissolved in 80 mL of an aqueous 1N-hydrochloric acid solution. While stirring the solution, 96 mL of aqueous ammonia (28% by weight) was added thereto at a rate of 2 mL/minute. Subsequently, the mixture was heated at 80° C. for 30 minutes and cooled to room temperature. The resulting agglomerate was purified by decantation with water. Generation of magnetite (Fe₃O₄) having a crystallite size of about 12 nm was observed by a X-ray diffraction method.

To the agglomerate was added 100 mL of an aqueous solution (adjusted to pH 6.8 with NaOH) in which 2.3 g of polyoxyethylene(4,5)lauryl ether acetate (Nikko Chemicals Co. Ltd.) was dissolved, and the solution was dispersed to give a magnetically responsive particle dispersion.

### Preparation Example 2

### Surface Modification of Magnetically Responsive Particle with Aspartic Acid

To 1.0 mL of the dispersion of magnetically responsive particles dispersed in water with a surfactant (polyoxyethylene(4,5)lauryl ether acetate) which was prepared in Preparation Example 1 (iron oxide content: 18.2 g/L) were added 1.0 mL of a 0.1 M phosphate buffer (pH 7.6) and 100 μl of a 1 M aspartic acid solution. The mixture was irradiated with ultrasonic wave in ultrasonic bath Sharp UT-105 at 100 W for 20 minutes. An agglomerated magnetic body was collected with a magnet and the supernatant was removed. Then, 2.0 mL of ethanol was added thereto, and the agglomerate was washed in a vortex mixer and collected again with the magnet, and the wash liquid was discarded. Subsequently, 2.0 mL of water was added thereto, the agglomerate was washed in the vortex mixer and collected again with the magnet, and the wash liquid was discarded. Lastly, 2.0 mL of water was added thereto and ultrasonic irradiation was performed at 100 W for 20 minutes. As a result, the magnetically responsive particles were homogeneously re-dispersed to form a transparent dispersion. When the zeta potential of the magnetically responsive particles was measured, the potential changed from −31 mV before measurement to −24 mV. This shows that substitution with aspartic acid occurred on the surface.

### Example 1

### Gelatin Nanoparticle Containing Adriamycin-Adsorbed Iron Oxide Particle

1.0 mL of the aspartic acid-modified magnetically responsive particle dispersion (Fe₃O₄ content: 1.0 mg/mL) prepared in Preparation Example 2 and an aqueous adriamycin solution (1.0 mg/mL) were mixed, and the resulting mixture was irradiated with ultrasonic wave at 100 W for 20 minutes using Ultrasonic bath Sharp UT-105. An agglomerated magnetic body was collected with a magnet and the supernatant was separated. The amount of remaining adriamycin (Abs. 480 nm) was measured from an absorption spectrum of the supernatant to calculate the amount of adriamycin immobilized on the magnetic body surface. Further, the magnetically responsive particle agglomerate separated using the magnet was re-dispersed by adding 1.0 mL of water in a vortex mixer. The amount of immobilized adriamycin was 200 μg/1.0 mg Fe₃O₄. The zeta potential changed from −24 mV to +17.7 mV, suggesting the presence of an amino group of adriamycin on the magnetic body surface.

0.2 mL of the aqueous dispersion of magnetically responsive particles, 20 mg of gelatin treated with lime, 1 mg of Daichitosan and 1.8 mL of ion exchange water are mixed. 1 mL of the resulting solution was poured into 10 mL of ethanol at an external temperature of 40° C. under a stirring condition of 800 rpm using a microsyringe. 2.5 μL of glutaraldehyde (20%) was added dropwise to the dispersion medium and the mixture was stirred for 30 minutes to give crosslinked gelatin nanoparticles. The particles have an average particle size of 135 nm as measured by a light scattering photometer, DLS-7000 made by OTSUKA ELECTRONICS CO., LTD.

### Example 2

### Albumin Nanoparticle Containing 5-Fluorouracil-Adsorbed Iron Oxide Particle

1.0 mL of the aspartic acid-modified magnetically responsive particle dispersion (Fe₃O₄ content: 1.0 mg/mL) prepared in Preparation Example 2 and an aqueous 5-fluorouracil solution (1.0 mg/mL) were mixed, and the resulting mixture was irradiated with ultrasonic wave at 100 W for 20 minutes using Ultrasonic bath Sharp UT-105. An agglomerated magnetic body was collected with a magnet and the supernatant was separated. The amount of remaining 5-fluorouracil (Abs. 254 nm) was measured from an absorption spectrum of the supernatant to calculate the amount of 5-fluorouracil immobilized on the magnetic body surface. Further, the magnetically responsive particle agglomerate separated using the magnet was re-dispersed by adding 1.0 mL of water in a vortex mixer. The amount of immobilized 5-fluorouracil was 200 μg/1.0 mg Fe₃O₄.

0.2 mL of the iron oxide nanoparticle dispersion, 20 mg of albumin, 1 mg of carboxymethylcellulose and 1.8 mL of ion exchange water are mixed. 1 mL of the resulting solution was poured into 10 mL of ethanol at an external temperature of 40° C. under a stirring condition of 800 rpm using a microsyringe. 2.5 μL of glutaraldehyde (20%) was added dropwise to the dispersion medium and the mixture was stirred for 30 minutes to give crosslinked albumin nanoparticles. The particles has an average particle size of 130 nm as measured by a light scattering photometer, DLS-7000 made by OTSUKA ELECTRONICS CO., LTD.

### Example 3

### Casein Nanoparticle Containing Astaxanthin-Adsorbed Iron Oxide Particle

1.0 mL of the aspartic acid-modified magnetically responsive particle dispersion (Fe₃O₄ content: 1.0 mg/mL) prepared in Preparation Example 2 and an ethanol-water mixed solution of astaxanthin (1.0 mg/mL) were mixed, and the resulting mixture was irradiated with ultrasonic wave at 100 W for 20 minutes using Ultrasonic bath Sharp UT-105. An agglomerated magnetic body was collected with a magnet and the supernatant was separated. The amount of remaining astaxanthin (Abs. 480 nm) was measured from an absorption spectrum of the supernatant to calculate the amount of astaxanthin immobilized on the magnetic body surface. Further, the magnetically responsive particle agglomerate separated using the magnet was re-dispersed by adding 1.0 mL of water in a vortex mixer. The amount of immobilized astaxanthin was 200 μg/1.0 mgFe₃O₄.

20 mg of casein is dissolved in 1.8 mL of a phosphate buffer at pH 10, and 0.2 mL of the iron oxide nanoparticle dispersion is added thereto. 1 mL of the resulting solution was poured into 10 mL of a phosphate buffer at pH 5 at an external temperature of 40° C. under a stirring condition of 800 rpm using a microsyringe, whereby casein nanoparticles were prepared. The particles have an average particle size of 135 nm as measured by a light scattering photometer, DLS-7000 made by OTSUKA ELECTRONICS CO., LTD.

### Example 4

### Gelatin Nanoparticle Containing Adriamycin-Adsorbed Iron Oxide Particle

Iron oxide particles were synthesized in the same manner as in Example 1. 0.2 mL of an iron oxide dispersion, 20 mg of acid-treated gelatin, 2 mg of chondroitin sulfate-C, 10 mg of transglutaminase and 1.8 mL of ion exchange water are mixed. 1 mL of the resulting solution was poured into 10 mL of ethanol at an external temperature of 40° C. under a stirring condition of 800 rpm using a microsyringe. The resulting dispersion was allowed to stand at an external temperature of 55° C. for 5 hours to give crosslinked gelatin nanoparticles. The particles have an average particle size of 85 nm as measured by a light scattering photometer, DLS-7000 made by OTSUKA ELECTRONICS CO., LTD.

### Example 5

### Albumin Nanoparticle Containing Adriamycin-Adsorbed Iron Oxide Particle

Iron oxide particles were synthesized in the same manner as in Example 1. Albumin is dissolved in a 0.5 M tris-hydrochloride buffer (pH 8.5) containing 3 mL of 7 M guanidine hydrochloride and 10 mM EDTA. Thereto was added 10 mg of dithiothreitol and the resultant was mixed and reduced at room temperature for 2 hours. The mixture was purified by gel filtration and 0.2 mL of the iron oxide dispersion was added to the resulting albumin solution. 1 mL of the resulting solution was poured into 10 mL of ethanol at an external temperature of 40° C. under a stirring condition of 800 rpm using a microsyringe. The resulting dispersion was stirred in the air at 40° C. for 3 hours to give crosslinked albumin nanoparticles. The particles have an average particle size of 180 nm as measured by a light scattering photometer, DLS-7000 made by OTSUKA ELECTRONICS CO., LTD.

### INDUSTRIAL APPLICABILITY

The nanoparticle of the present invention is highly safe because a polymer such as protein which is free of problems of biocompatibility is used. Also, since the nanoparticle of the present invention contains both magnetically responsive particles and a drug, the nanoparticle can be used simultaneously for imaging, hyperthermia and DDS. Furthermore, since an active substance is adsorbed to inorganic particles in the nanoparticle of the present invention, the nanoparticle is highly safe and has high drug incorporation ratio.

1. A nanoparticle which comprises an inorganic nanoparticle of 1 to 500 nm in average particle size having an active substance immobilized on the surface and a polymer.

2. The nanoparticle of claim 1 wherein the inorganic nanoparticle is a magnetic nanoparticle.

3. The nanoparticle of claim 1 wherein the inorganic nanoparticle is iron oxide, ferrite, zinc oxide, titanium oxide, silica or alumina.

4. The nanoparticle of claim 1 wherein an active substance is immobilized through physical adsorption on the surface of the inorganic nanoparticle having amino acid immobilized on the surface.

5. The nanoparticle of claim 1 wherein amino acid is immobilized on the surface of the inorganic nanoparticle surface-modified with a compound represented by the formula:
R¹—(OCH(R²)CH₂)ₙ—O-L-X
wherein R¹ represents an alkyl or alkenyl group having a carbon chain length between of 1 and 20 inclusive or an unsubstituted phenyl group or phenyl group substituted with an alkyl or alkoxyl group having a carbon chain length of 10 or less; R² represents a hydrogen atom or methyl group; n represents an integer of 1 to 20; L represents a single bond or an alkylene group having 1 to 10 carbon atoms; and X represents a carboxylic acid group, a phosphoric acid group, a sulfonic acid group or a boric acid group, and further, an active substance is immobilized through physical adsorption on the surface.

6. The nanoparticle of claim 1 which has an average particle size of 10 to 1000 nm.

7. The nanoparticle of claim 1 wherein the inorganic nanoparticle has an average particle size of 1 to 50 nm.

8. The nanoparticle of claim 1 wherein 0.1 to 100% by weight of the inorganic nanoparticle is contained with respect to the polymer.

9. The nanoparticle of claim 1 wherein 0.1 to 100% by weight of the active substance is contained with respect to the polymer.

10. The nanoparticle of claim 1 wherein the active substance is a cosmetic ingredient, a functional food ingredient or a pharmaceutical ingredient.

11. The nanoparticle of claim 10 wherein the cosmetic ingredient is a moisturizer, a skin-whitening agent or an anti-aging agent, the functional food ingredient is vitamin or an antioxidant, and the pharmaceutical ingredient is an anticancer agent, an antiallergic agent, an antithrombotic agent or an antiinflammatory agent.

12. The nanoparticle of claim 1 wherein the polymer is a synthetic polymer, a biodegradable polymer or a natural polymer.

13. The nanoparticle of claim 1 wherein the polymer is protein.

14. The nanoparticle of claim 1 wherein the protein is crosslinked during or after preparing the nanoparticle.

15. The nanoparticle of claim 14 wherein the protein is crosslinked by adding 0.1 to 100% by weight of a crosslinking agent with respect to the weight of the protein.

16. The nanoparticle of claim 15 wherein the crosslinking agent is an inorganic or organic crosslinking agent.

17. The nanoparticle of claim 16 wherein the crosslinking agent is enzyme, preferably transglutaminase.

18. The nanoparticle of claim 14 wherein the protein is crosslinked in an organic solvent.

19. The nanoparticle of claim 13 wherein the protein has a lysine residue and a glutamine residue.

20. The nanoparticle of claim 13 wherein the protein is collagen, gelatin, albumin, ovalbumin, casein, transferrin, fibrin, fibrinogen or a mixture thereof.

21. The nanoparticle of claim 13 wherein the protein is acid-treated gelatin or albumin.

22. The nanoparticle of claim 13 wherein the protein is acid-treated gelatin, and the nanoparticle is prepared by crosslinking the acid-treated gelatin with an enzyme during or after preparing the nanoparticle comprising the inorganic nanoparticle and the acid-treated gelatin.

23. The nanoparticle of claim 13 which is produced through the following steps:
(a) mixing a solution of at least one active substance and inorganic nanoparticles, thereby adsorbing the active substance to the surface of the inorganic nanoparticles;
(b) dissolving protein in an aqueous medium;
(c) mixing the inorganic nanoparticles to which at least one active substance is adsorbed and the protein solution;
(d) pouring the solution prepared in step (c) into an organic solvent; and
(e) crosslinking the protein by adding a crosslinking agent.

24. The nanoparticle of claim 13 which is produced through the following steps:
(a) mixing a solution of at least one active substance and inorganic nanoparticles, thereby adsorbing the active substance to the surface of the inorganic nanoparticles;
(b) dissolving protein in an aqueous medium;
(c) mixing the inorganic nanoparticles to which at least one active substance is adsorbed and the protein solution;
(d) adding an enzyme; and
(e) pouring the solution prepared in step (d) into an organic solvent to crosslink the protein with the enzyme.

25. The nanoparticle of claim 14 which is obtained by treating protein with a reducing agent to break a disulfide bond in protein molecules, then forming nanoparticles of the protein, and further treating the protein with an oxidant.

26. The nanoparticle of claim 25 wherein, in the step of treating protein with an oxidant, protein nanoparticles dispersed in an organic solvent are treated with the oxidant.

27. The nanoparticle of claim 25 wherein the protein is albumin, ovalbumin, transferrin or globulin.

28. The nanoparticle of claim 25 which is produced through the following steps:
(a) mixing a solution of at least one active substance and inorganic nanoparticles, thereby adsorbing the active substance to the surface of the inorganic nanoparticles;
(b) dissolving protein whose disulfide bond is reduced in water;
(c) mixing the inorganic nanoparticles to which at least one active substance is adsorbed and the protein solution;
(d) pouring the solution prepared in step (c) into an organic solvent; and
(e) treating the resultant with an oxidant.

29. The nanoparticle of claim 13 wherein the protein is casein.

30. The nanoparticle of claim 29 which is produced through the following steps:
(a) mixing a solution of at least one active substance and inorganic nanoparticles, thereby adsorbing the active substance to the surface of the inorganic nanoparticles;
(b) dissolving casein in a basic aqueous medium at pH 8 or more;
(c) mixing the inorganic nanoparticles to which at least one active substance is adsorbed and the casein solution; and
(d) pouring the solution prepared in step (c) into an aqueous medium at pH 3.5 to 7.5.

31. The nanoparticle of claim 29 which is produced through the following steps:
(a) mixing a solution of at least one active substance and inorganic nanoparticles, thereby adsorbing the active substance to the surface of the inorganic nanoparticles;
(b) dissolving casein in a basic aqueous medium at pH 8 or more;
(c) mixing the nanoparticles to which at least one active substance is adsorbed and the casein solution; and
(d) lowering the pH of the solution prepared in step (c) to pH 3.5 to 7.5 while stirring.

32. The nanoparticle of claim 1 wherein 0.1 to 100% by weight of lipid is added with respect to the weight of the polymer.

33. The nanoparticle of claim 1 wherein 0.1 to 100% by weight of a cationic or anionic polysaccharide is added with respect to the weight of the polymer.

34. The nanoparticle of claim 1 wherein 0.1 to 100% by weight of a cationic or anionic protein is added with respect to the weight of the polymer.

35. The nanoparticle of claim 1 wherein 0.1 to 100% by weight of cyclodextrin is added with respect to the weight of the polymer.

36. A hyperthermia agent which comprises the nanoparticle of claim 1.

37. An MRI contrast medium which comprises the nanoparticle of claim 1.

38. A drug delivery agent which comprises the nanoparticle of claim 1.

39. A method for producing a nanoparticle comprising an inorganic nanoparticle of 1 to 500 nm in average particle size having an active substance immobilized on the surface, and a protein, the method comprising crosslinking the protein during and/or after preparing the nanoparticle.

40. The method for producing a nanoparticle according to claim 39, wherein the protein is crosslinked by an enzyme.

41. The method for producing a nanoparticle according to claim 40, wherein the protein is crosslinked by the enzyme in an organic solvent.

42. A method for producing a nanoparticle comprising an inorganic nanoparticle of 1 to 500 nm in average particle size having an active substance immobilized on the surface, and a protein, the method comprising treating the protein with a reducing agent to break a disulfide bond in protein molecules, then forming nanoparticles of the protein, and further treating the protein with an oxidant.

43. A method for producing a nanoparticle comprising an inorganic nanoparticle of 1 to 500 nm in average particle size having an active substance immobilized on the surface, and a protein, the method comprising treating the protein with a reducing agent to break a disulfide bond in protein molecules, then forming nanoparticles of the protein, and further treating the protein dispersed in an organic solvent with an oxidant.

44. A method for producing a nanoparticle of 10 to 1000 nm in average particle size, comprising an inorganic nanoparticle of 1 to 500 nm in average particle size having an active substance immobilized on the surface and casein, the method comprising the following steps:
(a) mixing a solution of at least one active substance and inorganic nanoparticles, thereby adsorbing the active substance to the surface of the inorganic nanoparticles;
(b) dissolving casein in a basic aqueous medium at pH 8 or more;
(c) mixing the inorganic nanoparticles to which at least one active substance is adsorbed and the casein solution; and
(d) pouring the solution prepared in step (c) into an aqueous medium at pH 3.5 to 7.5.

45. A method for producing a nanoparticle of 10 to 1000 nm in average particle size, comprising an inorganic nanoparticle of 1 to 500 nm in average particle size having an active substance immobilized on the surface and casein, the method comprising the following steps:
(a) mixing a solution of at least one active substance and inorganic nanoparticles, thereby adsorbing the active substance to the surface of the inorganic nanoparticles;
(b) dissolving casein in a basic aqueous medium at pH 8 or more;
(c) mixing the inorganic nanoparticles to which at least one active substance is adsorbed and the casein solution; and
(d) lowering the pH of the solution prepared in step (c) to pH 3.5 to 7.5 while stirring.
